(19) United States
(12) Plant Patent Application Publication
(10) Pub. No.: US 2013/0247256 P1
(43) Pub. Date: Sep. 19, 2013

(54) Orange tree named 'Kirkwood Red'

(50) ;

(71) Applicant: André Potgieter, Kirkwood (ZA)

(72) Inventor: André Potgieter, Kirkwood (ZA)

(73) Assignee: Kirkwood Red Trust, Kirkwood (ZA)

(21) Appl. No.: 13/385,922

(22) Filed: Mar. 13, 2012

(51) Int. Cl. A01H 5/00

(52) U.S. Cl. PLT/202

(57) ABSTRACT

A new and distinct variety of sweet orange tree (Citrus sinensis) named ‘Kirkwood Red.’ ‘Kirkwood Red’ is notable for its fruit, which has an attractive external blush, dark red flesh, and exceptional taste.

### CROSS-REFERENCE TO RELATED APPLICATIONS

None

### GENUS AND SPECIES

Citrus sinensis

### VARIETY DENOMINATION

‘Kirkwood Red’

### BACKGROUND AND SUMMARY OF THE VARIETY

The new orange tree ‘Kirkwood’ originated as a whole tree mutation of a ‘Palmer’ navel orange tree (not patented). The mutation was discovered in 1992 in a commercial orchard of ‘Palmer Navel’ trees grown on ‘Swingle’ citrumelo and rough lemon rootstock (not patented) at Kirkwood, South Africa, and was distinguished by its external blush, dark red flesh, and excellent flavor. Asexual propagation by budding was carried out at Kirkwood to determine whether the desirable characteristics of the mutation would carry through to asexually propagated progeny. It has been found that the desirable characteristics are in fact reproduced through asexual propagation, and remain stable through successive generations.

‘Kirkwood Red’ is a new and distinct orange tree notable for its fruit, which is distinguished by its external blush, dark red flesh, and exceptional flavor. The ‘Kirkwood Red’ tree is distinguished by red pigmentation present in vascular bundles of the leaves and the fruit stem.

### BRIEF DESCRIPTION OF THE PHOTOGRAPHS

FIG. 1 shows whole and sectioned fruit of the ‘Kirkwood Red’ variety;

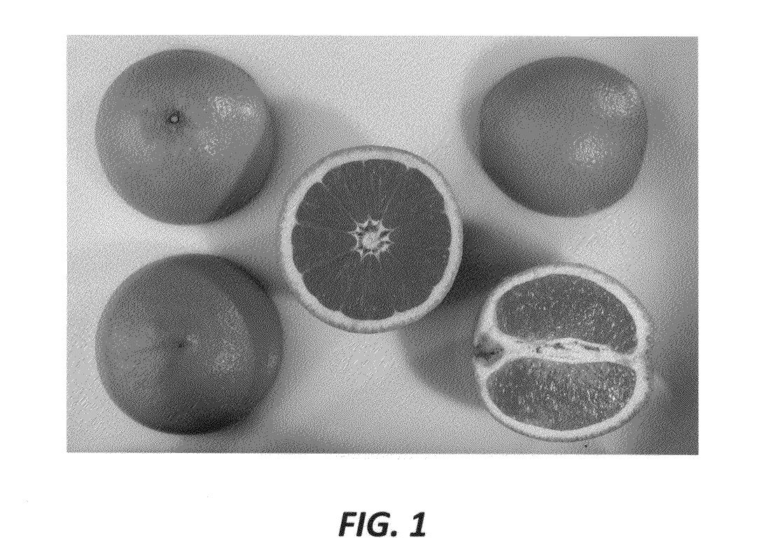

FIG. 2 shows a ‘Kirkwood Red’ tree on ‘Carrizo’ citrange rootstock;

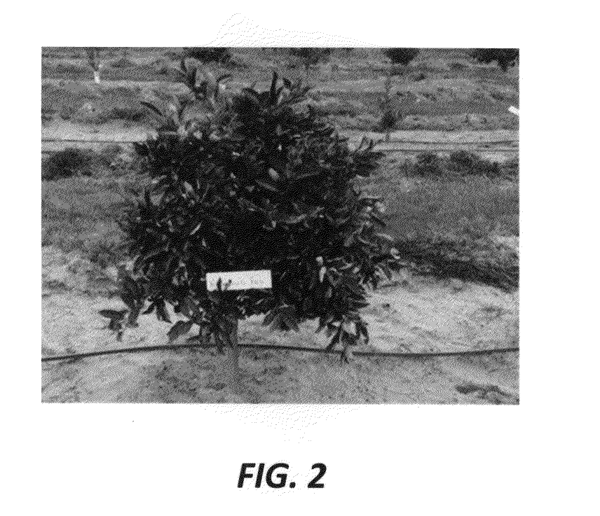

FIG. 3 shows a branch and leaves of the ‘Kirkwood Red’ variety;

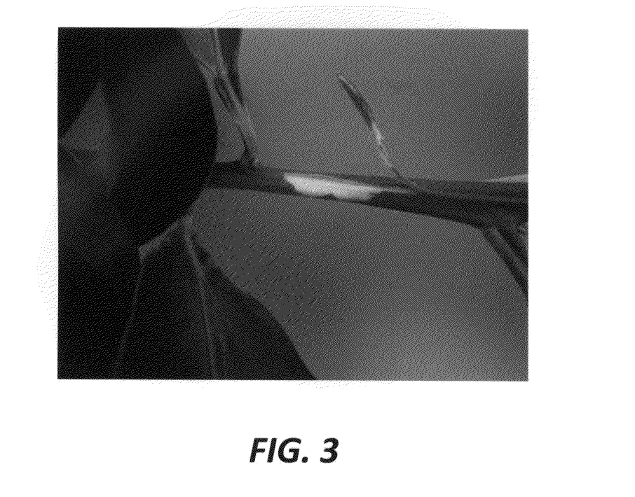

FIG. 4 shows the red pigmentation on the stalk end of the ‘Kirkwood Red’ variety; and

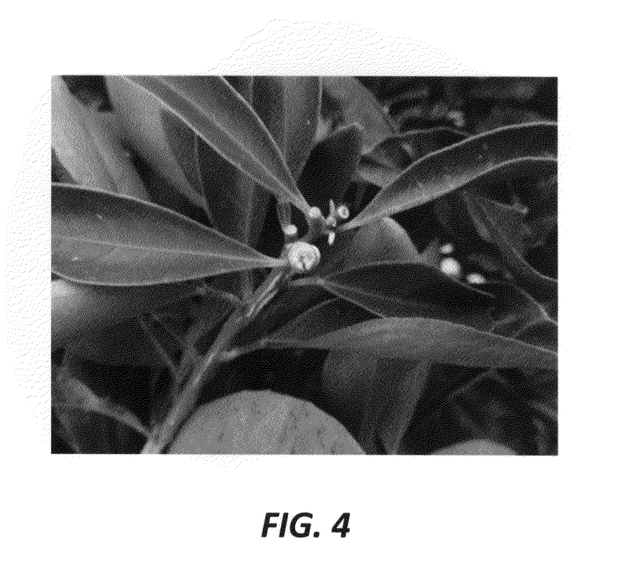

FIG. 5 shows the red pigmentation on the calyx and fruit of the ‘Kirkwood Red’ variety.

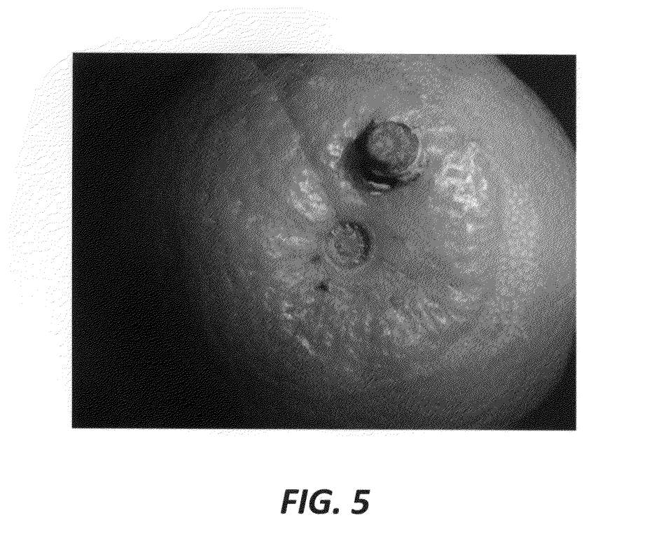

The colors of the claimed variety may vary with lighting conditions. Color characteristics of the variety should therefore be determined with reference to the observations described herein, rather than from these illustrations alone.

### DETAILED BOTANICAL DESCRIPTION

The following detailed botanical description is based on observations of the original tree of the variety, planted in 1994 and grown on ‘Swingle’ citrumelo and rough lemon rootstock. Observations were recorded and photographs taken during the 2010 growing season at Kirkwood, South Africa. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average. Colors are described with reference to The Royal Horticultural Society Colour Chart (2007).
Tree:
   Vigor.—Comparable to ‘Washington’ navel (not patented).
   Density of canopy.—Compact.
   Habit.—Comparable to ‘Washington’ navel.
   Height.—1.5 meters.
   Spread.—1.7 meters.
   Trunk diameter.—43 mm (30 cm above the graft).
   Bark color.—Brown N200C.
   Bark texture.—Smooth.
   Tendency to alternate bearing.—None.
   Winter hardiness.—Slightly more sensitive than average navel.
Branches:
   Length.—250 mm.
   Diameter.—11 mm.
   Crotch angle.—45° maximum.
   Color.—Green 136C with Red 50D vascular bundle.
   Bark texture.—Smooth.
   Thorns.—Present on some branches, generally close to trunk and vigorous branches
   Thorn length.—10 mm.
   Current year shoot length.—120 to 200 mm (latest flush).
   Current year shoot color.—Light green.
Flowers:
   Bud shape.—Elongated.
   Bud length.—12.6 mm.
   Bud diameter.—7.7 mm.
   Bud color.—White.
   Quantity of blossoms per cluster.—Up to 8.
   Blossom diameter.—44 mm (open to ends of petals).
   Blossom depth.—22 mm base of sepal to top of petal.
   Pollen present.?— Not seen.
   Sepal length.—4.9 mm.
   Sepal width.—4.9 mm.
   Sepal shape.—Acuminate.
   Sepal margin.—Entire.
   Sepal color — upper surface.—White with tinge of light green.
   Sepal color — lower surface.—White with tinge of light green.
   Quantity of petals per flower.—4 to 6, generally 5.
   Petal shape.—Stellate.
   Petal apex.—Acute.
   Petal margin.—Entire (smooth).
   Petal length.—21 mm.
   Petal width.—6.5 mm.
   Petal color — upper surface.—White.
   Petal color — lower surface.—White with yellow dots.
   Date of first bloom.—12 October.
   Date of full bloom.—15 October.
   Date of first fruitlet fall.—around 21 November.
   Pedicel length.—6.0 mm.
   Pedicel diameter.—1.7 mm.
   Pedicel color.—Cream to light green.
   Pistil quantity per flower.—One.
   Pistil length.—13.5 mm.
   Pistil color.—Light green, yellow/cream.
   Anther quantity per flower.—24.
   Anther length.—1.48 mm.
   Anther color.—Yellow brown.
   Stigma quantity per flower.—One.
   Stigma length.—1.97 mm.
   Stigma color.—Cream.
   Style quantity per flower.—One.
   Style length.—6.97 mm.
   Style color.—Very light green.
   Ovary quantity per flower.—One.
   Ovary length.—3.30 mm.
   Ovary diameter.—4.56 mm.
   Ovary color.—Green.
Leaves (from twigs behind latest flush):
   Length.—70 mm.
   Width.—32 mm.
   Length/width ratio.—1:2.2.
   Blade margin.—Entire to very slightly undulate.
   Blade shape.—Elliptic, involute.
   Apex shape.—Acute, emargination at tip.
   Base shape.—Cuneate.
   Color — upper surface.—Green 136B.
   Color — lower surface.—Green 136C.
   Petiole length.—12 mm.
   Petiole diameter.—1.8 mm.
   Petiole color.—Green 145A; vascular bundles are red 50C.
   Leaf texture.—Smooth.
   Wings.—Present.
   Wing width.—3 mm.
Fruit:
   Quantity per cluster.—2 to 3.
   Axial diameter.—73 to 81 mm.
   General shape in profile.—Round to slightly oval.
   Navel.—Small navel end, sometimes closed.
   Rind color.—Deep orange to light red N25A, N25D, N30A.
   Rind thickness.—6 mm.
   Rind texture.—Medium smooth to smooth; weak to medium strong gloss.
   Ease of peeling.—Good peelability.
   Albedo.—White to light yellow with some pink staining.
   Toughness of segment membrane.—Medium.
   Juice sac color.—Orange-red N30C.
   Juice sac length.—8 mm.
   Juice sac shape.—Elongated.
   Juice soluble solids (Brix).—10.7° to 11.5°.
   Quantity of segments per fruit.—Generally 9 to 11.
   Seeds.—None.
   Relative harvest maturity.—Mid-season.
   Harvest window.—June.
   Harvest yield.—38.9 kg/tree (4 year old topworks).
   Market use.—Fresh.

1. A new and distinct variety of orange tree, substantially as illustrated and described herein.
